(19) United States
(12) Patent Application Publication
(10) Pub. No.: US 2010/0331769 A1
(43) Pub. Date: Dec. 30, 2010

(54) NOZZLE FOR HIGH-SPEED JETTING DEVICES

(75) Inventors: Giovanni Nisato, Eindhoven (NL); Freddy Roozeboom, Eindhoven (NL); Jan-Eric Jack Martijn Rubingh, Eindhoven (NL); Wouter Dekkers, Eindhoven (JP)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/446,468

(22) PCT Filed: Oct. 23, 2007
(86) PCT No.: PCT/IB07/54295

§ 371 Date: Apr. 21, 2009

(30) Foreign Application Priority Data
Oct. 25, 2006 (EP) 06122942.3

(51) Int. Cl. A61M 3/02; B23P 17/00; B05B 1/00

(52) U.S. Cl. 604/39; 29/890.09; 239/589

(57) ABSTRACT

A nozzle for jetting devices is described comprising e.g. one patterned silicon substrate enabling semiconductor mass production. The method of manufacturing the nozzle is characterized by using one mask layer deposited on the silicon substrate. The etching of the silicon substrate is done by means of a first isotropic etching step and a second anisotropic etching step through the mask layer, resulting in a perfectly aligned nozzle.

### FIELD OF THE INVENTION

The current invention is related to a method of manufacturing a nozzle for high-speed jetting devices for the ejection of a fluid, and to a nozzle for high-speed jetting devices.

### BACKGROUND OF THE INVENTION

In U.S. Pat. No. 3,921,916, a method of manufacturing nozzles in monocrystalline silicon is disclosed. The method utilizes anisotropic etching through a silicon substrate to an integral etch-resistant barrier layer doped heavily with P-type impurities. The P-type layer is then etched through at the bottom of the previously etched structure to form a hole. The nozzle manufactured by this method comprises a nozzle body formed of a semiconductor material having a rectangular entrance aperture of a first cross-sectional area which tapers to a second cross-sectional area which is smaller than the cross-sectional area of said entrance aperture; and a membrane of said semiconductor material, formed within said second cross-sectional area and having an exit aperture formed therein having a smaller cross-sectional area than said second cross-sectional area and having a different cross-sectional geometry than said second cross-sectional area. The difficulty of this method is to provide good alignment of the first etching step and the second etching step. Misalignment is not critical as long as the nozzle is used in low-speed jetting applications with a fluid ejection speed below 10 m/s as in e.g. ink-jet printing. In high-speed jetting applications with a fluid ejection speed above 60 m/s misalignment can cause turbulent flow, which decreases the ejection efficiency of the high-speed jetting device, said turbulent flow being characterized by means of the relation between input energy and maximum available fluid ejection speed.

### SUMMARY OF THE INVENTION

It is an object of the current invention to provide an improved method of manufacturing a nozzle for the jetting of fluids.

The object is achieved by means of a method comprising the steps of:
providing a substrate having a first side and a second side;
providing a mask layer on the first side of the substrate and a protection layer (3) on the second side of the substrate;
providing at least one opening in the mask layer;
forming an ejection cavity by removing parts of the substrate isotropically through the opening in the mask layer;
forming at least one ejection tube (30) by removing parts of the substrate (1) anisotropically through the opening (10) in the mask layer (2),
opening the ejection tube at the second side of the substrate (1) and
removing the mask layer (2).

Removing parts of the substrate isotropically means that the maximum width of the ejection chamber, being measured parallel to the first side of the substrate, is independent of the width of the opening in the mask layer. Examples are e.g.:
Isotropic etching through the opening in the mask layer, resulting in a hemispherical cavity if effects at the interface between substrate and mask layer can be neglected, and the substrate has an isotropic structure.
Etching of the substrate through the opening in the mask layer, such that the etching velocity at the interface between the mask layer and the substrate is higher than the etching velocity in the substrate.
Vaporization of the substrate material (e.g. a polymer) by means of heating up the opening in the mask layer and the surrounding area of the opening in the mask layer

Removing parts of the substrate anisotropically means that the maximum width of the ejection chamber, being measured parallel to the first side of the substrate, is determined by the width of the opening in the mask layer. Examples are e.g.:
Anisotropic etching through the opening in the mask layer, resulting in a tube in the substrate with a diameter determined by the width of the opening in the mask layer.
Laser drilling of the substrate by using the opening in the mask layer as a kind of shadow mask.

The order of the steps is not fully determined. The ejection tube can be formed in a first step by means of e.g. anisotropic etching of the substrate and the ejection chamber can be formed in a subsequent step by using a second etchant being characterized by a higher etching velocity at the interface between the mask layer and the substrate in comparison with the isotropic etching velocity in the substrate. In an alternative approach, the ejection chamber is formed in a first step by means of isotropic etching of the substrate through the opening in the mask layer and the ejection tube is formed by means of a subsequent anisotropic etching step.

Further, there are different approaches to forming the ejection tube and opening the ejection tube at the second side of the substrate:
The ejection tube is etched anisotropically in a first step down to the protective layer. The protective layer is removed in a subsequent step.
The ejection tube is etched anisotropically in a first step but the protective layer is not reached. The protective layer is removed in a second step and the substrate is thinned down subsequently by e.g. grinding or etching the second side of the substrate until the ejection tube is opened.
The protective layer is removed and the substrate is thinned down subsequently by e.g. grinding or etching the second side of the substrate. Finally, the substrate is removed in an anisotropic way through the mask layer until the second side of the substrate is reached and the ejection tube is opened.

All approaches guarantee a perfect alignment of ejection chamber and ejection tube or ejection tubes, since the opening(s) in the mask layer is/are used for forming the ejection chamber as well as for forming the ejection tube(s). Further, the method according to the present invention can be used in such a way that no corners are present in the ejection chamber if only one circular opening in the mask layer is used to provide the ejection chamber and the ejection tube. Additionally, unlike the prior art, there is no step-like transition between ejection chamber and ejection tube. Both measures reduce turbulent flow, thus improving the ejection efficiency. The method has the additional advantage that an array of nozzles can be manufactured easily.

A subsequent step of removing parts of the substrate from the first side of the substrate can be added to the method. The parts of the substrate are removed by means of etching or grinding the first side of the substrate. This subsequent step can be used to thin the substrate. In addition, the substrate can be removed to the extent that the maximum width of the ejection chamber is reached. This measure results in a tapered ejection chamber in the case that the mask layer (before it is removed) and the remaining substrate material enclose an angle of less than 90° (taking the tangent to the boundary of the ejection chamber at the point where the remaining substrate touches the mask layer), thus accelerating the fluid to be ejected and improving the fluid dynamics of the fluid to be ejected, which is favorable for high-speed jetting.

In a further embodiment of a method according to the invention, the surface of the ejection chamber and the ejection tube is smoothened by removing the surface of the ejection chamber and the ejection tube superficially. There are two different approaches:

i) The surface of the substrate is oxidized after ejection chamber and ejection tube have been provided (e.g. by heating the substrate in an oxidizing atmosphere). Based on the precondition that the substrate material and the oxide of the substrate material (e.g. Si substrate oxidized to SiO₂ superficially) can be removed selectively (SiO₂ e.g. by HF), the oxide layer is removed resulting in a smoothened surface since the relation between surface and volume of structures defining the roughness of the surface results in faster oxidization rates of these structures. Consequently, removing the oxidized substrate material results in reduced roughness of the surface. Additional mask steps can be used to limit the smoothening procedure to the ejection chamber and the ejection tube.

ii) The surface of the substrate can be directly removed by means of isotropic etching. Using a highly selective etchant such as e.g. XeF₂ in the case of a silicon substrate reduces the roughness of a surface, since the relation between surface and volume of structures defining the roughness of the surface of the substrate results in faster etching of this structures. Additional mask steps can be used to limit the smoothening procedure to the ejection chamber and the ejection tube.

The smoothened surface of the ejection chamber and the ejection tube reduces friction with the fluid to be ejected. In addition, edges at e.g. the etch transition between ejection chamber and ejection tube can be smoothened, thereby reducing or even preventing turbulent flow of the fluid to be ejected, resulting in an increased ejection efficiency. An additional measure to smoothen the surface of the ejection chamber and the ejection tube is the provision of a smoothening layer. Taking e.g. a silicon substrate, a smoothening layer can be provided by e.g. low-pressure chemical vapor deposition (LPCVD) of phosphorous silicate glass (PSG) or borophosphosilicate glass (BPSG) and a subsequent reflow step (PSG: between 950° C. and 1100° C.; BPSG: around 800° C.). Other methods to provide the smoothening layer are e.g. dip coating, spray coating and the like.

In a further embodiment of a method according to the invention, the smoothening layer is adapted to the fluid to be ejected through the nozzle. The smoothening layer reduces surface defects that can cause bubble formation, especially when large pressures are applied to the fluid which can lead to cavitation and loss of jet velocity. The material of the smoothening layer has an angle of contact of less than 90° with the fluid to be ejected. The fluid to be ejected wets the surface of the ejection chamber and the ejection tube. This is advantageous if the nozzle is attached to a jetting device and self-filling of the ejection chamber with the fluid to be ejected after a jet is ejected is wanted. The ejection chamber and ejection tube are fully filled with the fluid to be ejected especially if the angle of contact between the smoothening layer and the fluid to be ejected is near to zero (fully wetting). Taking e.g. a water-based fluid to be ejected, a hydrophilic smoothening layer of e.g. PSG can be provided as described above. Alternatively, a polymer like parylene can be deposited by means of LPCVD usually at room temperature. Other hydrophobic coatings (e.g. octadecyl-trichlorosilane, trimethoxy(3,3,3 trifluoropropyl)silane) can also be used to enable the jetting of oil-based fluids to be ejected.

Inorganic coatings with biocidal properties (e.g. AgCl, AgBr) or polymer coatings containing (nano)particles of biocidal salts can be applied to reduce the risk of bacterial growth and contamination (ref: J. Am. Chem Soc. 2006, 128, 9712).

In another embodiment of a method according to the invention, a termination layer is provided on the second surface of the substrate, which termination layer has an angle of contact with the fluid to be ejected of more than 90°. The termination layer is not wetted by the fluid to be ejected. Taking e.g. a water-based fluid to be ejected, a hydrophobic layer of silane compounds (e.g. octadecyl-trichlorosilane) or fluorinated compounds such as trimethoxy(3,3,3 trifluoropropyl)silane can be provided. The hydrophobic coating promotes droplet formation at the second surface of the substrate, especially in low-speed dispensing regimes, and prevents the formation of fluid films on the outer layer of the nozzle plate, which would hinder the jet formation.

In a further embodiment of a method according to the invention, a further indentation structure is provided in the first side of the substrate. The indentation structure is provided by means of an indentation opening in the mask layer. The indentation opening enables to provide the indentation structure in the substrate as soon as the ejection chamber and the ejection tube are provided. The indentation structure can e.g. be a concentric circular groove around the ejection chamber and the ejection tube. With respect to the small size of the ejection chamber and the ejection tube, the effort to detect and localize the ejection chamber and the ejection tube can be reduced by the indentation structure. Further, the indentation structure can be used to align the nozzle to the housing of a jetting device in order to optimize the fluid flow. If parts of the substrate are removed from the first side, the depth of the indentation structure has to be adapted accordingly. In addition or alternatively, an alignment structure can be provided to the first side of the substrate. This alignment structure can e.g. be a layer deposited and structured on the first side of the substrate in order to form a kind of key fitting a complementary structure of the high-speed jetting device. It can also be e.g. a structured plating base for e.g. copper. When depositing copper by galvanic processing (e.g. electroless plating), a structured base is provided that can be used to solder the nozzle to an essentially identical structure attached to the housing of the jetting device. The self-alignment of the nozzle during the soldering simplifies the assembly of the jetting device.

It is a further object of the current invention to provide an improved nozzle for the jetting of fluids.

This object is achieved by means of a nozzle for the jetting of fluids comprising:
a substrate with a first side and a second side;
an ejection chamber being a cavity in the substrate and being open on the first side of the substrate, the cross-section of the ejection chamber parallel to the first side of the substrate being not constant as a function of depth;
at least one ejection tube being a cavity in the substrate and being open on the second side of the substrate, the cross-section of the ejection tube (30) parallel to the first side of the substrate being constant as a function of depth, and
the ejection chamber and the at least one ejection tube are connected with each other, forming a passage through the substrate.

For high-speed jetting it is advantageous that the maximum cross-sectional area of the ejection chamber parallel to the first side of the substrate is located in the plane defined by the first side of the substrate. Further it is advantageous for high-speed jetting if the cross-sectional area of the ejection chamber parallel to the first side of the substrate is tapered starting in the plane defined by the first side of the substrate. In addition it is advantageous for high-speed jetting if the ejection tube preferably has a constant cross-sectional area parallel to the first side of the substrate. Limited tapering of the ejection tube is a result of process variations during the production of the nozzle well known to those skilled in the art.

The nozzle can either be used for low-speed jetting such as e.g. in ink jet printers or for transdermal drug delivery where drugs are injected through the skin and high fluid speeds above 60 m/s are needed in order to penetrate the multiple layers constituting the (human) skin.

Further application areas are oral health care devices utilizing high-speed (water) jets for removing bio films on teeth or gum.

In one embodiment of a nozzle according to the invention, the ejection tube is cylindrical, the ejection chamber is an hemispherical cavity, and the ejection chamber and the ejection tube are aligned along the cylinder axis of the ejection tube. This highly symmetric configuration of the nozzle prevents edges causing turbulent flow from reducing the ejection efficiency like the pyramid-shaped nozzles described in the prior art. The pyramid shape of the nozzle with a round opening in the etch-resistant barrier layer, as disclosed in U.S. Pat. No. 3,921,916, is well suited for low-speed jetting applications with a fluid ejection speed below 10 m/s, such as e.g. ink-jet printing. In high-speed jetting devices with a fluid ejection speed above 60 m/s, the pyramid shape and the discontinuous etching transition to the round opening causes a highly turbulent flow, resulting in satellite jets ejected through the opening. The satellite jets dramatically decrease the ejection efficiency of the high-speed jetting device, said ejection efficiency being characterized by means of the relation between input energy and maximum available fluid ejection speed.

In another embodiment of the current invention, the nozzle as described above is part of a jetting device, the jetting device further comprising a power supply, a pressure applicator applying pressure to a fluid to be ejected through the nozzle, the flow direction of the fluid to be ejected during ejection being from the ejection chamber to the ejection tube. The nozzle is assembled to the jetting device in a way that the fluid to be ejected enters the nozzle through the ejection chamber and leaves the jetting device via the ejection tube. The second side of the substrate is part of the outer surface of the jetting device. The continuous and smooth tapering of the ejection chamber and the smooth transition to the ejection tube suppress turbulent flow causing unwanted losses. The pressure applicator can be an electrostatically actuated piston or membrane integrated in a housing, a thermally actuated piston or membrane integrated in a housing or a piezoelectrically actuated piston or membrane integrated in a housing. Further components that can be added to the jetting device are a fluid chamber increasing the volume of the ejection chamber, a fluid reservoir, a supply pipe connecting the fluid chamber and the ejection chamber with the fluid reservoir and means to control the ejection, such as integrated circuitry and sensors.

The jetting device can either be used for low-speed jetting such as e.g. in ink jet printers or for transdermal drug delivery where drugs are injected through the skin and high fluid speeds of above 60 m/s are needed in order to penetrate the multiple layers constituting the (human) skin. Further application areas are oral health care devices utilizing high-speed (water) jets for removing bio films on teeth or gum.

### BRIEF DESCRIPTION OF THE DRAWINGS

The present invention will be explained in greater detail with reference to the Figures, in which the same reference signs indicate similar parts, and in which:

FIGS. 1 to 9 are cross-sectional views illustrating various consecutive steps of a method of manufacturing a nozzle.

FIG. 10 shows a cross-sectional view of one embodiment of the nozzle with alignment structure.

FIGS. 11 and 12 are cross-sectional views illustrating further method steps to smoothen the surface of the nozzle.

FIGS. 13 to 15 are top views of different designs of mask layers.

FIG. 16 shows a SEM picture of a cross section of a nozzle manufactured by means of a method according to the invention.

FIG. 17 is a cross-sectional view of a high-speed jetting device with a nozzle according to the current invention.

FIG. 18 is a cross-sectional view of a further high-speed jetting device with a nozzle according to the current invention.

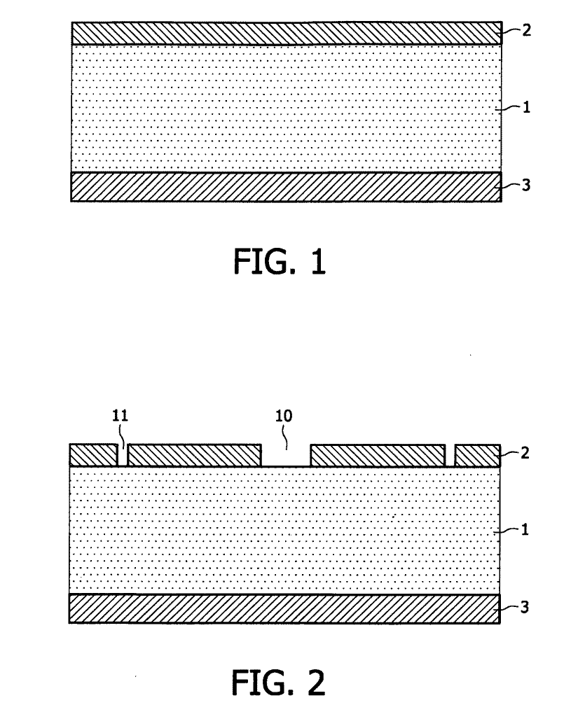

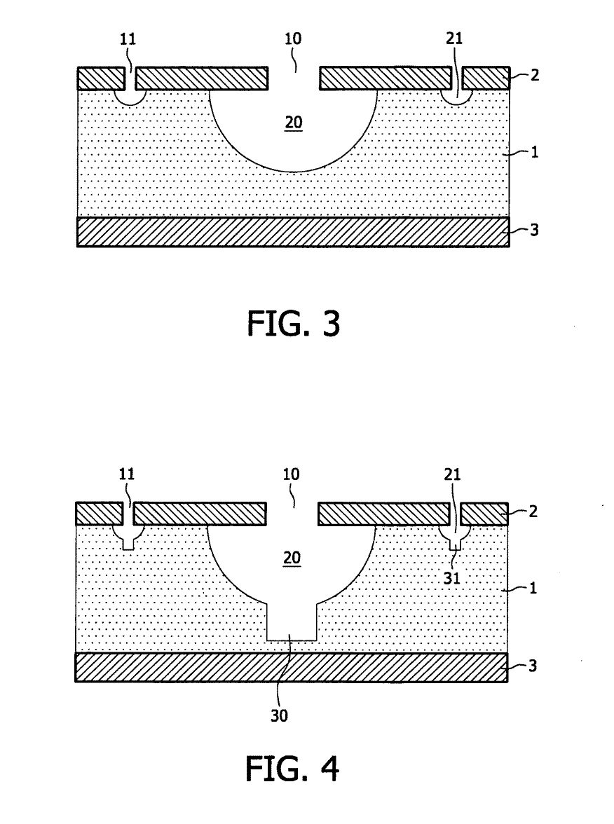

FIG. 1 shows a principal sketch of a cross-section of a silicon substrate 1 with a first side and a second side. A mask layer 2 of silicon oxide is deposited as a hard mask on the first side of the silicon substrate 1. The second side of the silicon substrate is coated with a photoresist building a protection layer 3. In FIG. 2 the lithographic patterning of the mask layer 2 is shown. A round opening 10 with a diameter of typically 10 μm-200 μm and a ring-like narrow indentation opening 11 with a width of typically 1 μm-3 μm concentrically arranged around the opening 10 are etched in the silicon oxide mask layer 2. In FIG. 3 the silicon substrate 1 with patterned mask layer 2 and the protection layer 3 are shown after isotropic dry etching of the silicon substrate through the opening 10 and the indentation opening 11 by means of SF₆ etching gas only in the etch reaction chamber and without applying a bias voltage to the wafer chuck. Etching through the round opening 10 in the silicon oxide mask layer 2 results in an essentially hemispherical cavity with a diameter of about 100 μm up to several 100 μm being the ejection chamber 20 in the silicon substrate 1. Etching through the narrow indentation opening 11 results in a circular trench 21 extending concentrically around the ejection chamber 20 with a semicircle-like cross-sectional area. The depth of the isotropic etching is controlled by means of the width of the opening 10 and the indentation opening 11 in the mask layer and the etching time. The wider the openings are, the larger the etching depth is. Here, the final width of the opening (and thus the depth) of the circular trench 21 is determined by the extent of post-process grinding and polishing of the first side of the substrate 1: after this thinning, the circular trench 21 should still be present/visible to facilitate the singulation of the entire disk-shaped orifice. By means of these etching steps further channels can be provided on the first surface of the silicon substrate. These channels can e.g. be used to provide a connection between the ejection chamber 20 and a fluid reservoir in a high-speed jetting device. In the following processing step shown in FIG. 4, the silicon substrate is etched anisotropically by switching to (anisotropic) Bosch-etch conditions. This is brought about by time-multiplexed, alternate introduction of SF₆/O₂ and C₄F₈ gas into the plasma. The SF₆/O₂ gas etches the pores and the C₄F₈ gas forms a Teflon-like passivation layer on the pore walls until the desired depth of, say, a few tens of μm of the anisotropically etched pores, is reached. Here, the Bosch process is characterized by the use of a bias voltage on the wafer chuck, such that the etching takes place mainly in the Reactive Ion Etching regime, yielding a pore diameter nearly identical to the diameter of the opening 10 in the mask layer 2. Anisotropic etching finally results in a cylindrical ejection tube 30 at the bottom of the ejection chamber 20 and a circular trench 31 with a rectangular cross section perpendicular to the first side of the substrate 1 at the bottom of the circular trench 21. Both the circular trench 21 and the circular trench 31 build the indentation structure and should be visible even after removal of parts of the substrate 1 from the first side of the substrate 1.

The shape of the tube 30 (and the circular trench 31) can be further tuned towards a tapered (rather than cylindrical) profile by ramping up the C₄F₈ passivation gas concentration or the passivation cycle time, or ramping down the voltage applied to the bias voltage chuck during the passivation cycles. Here, the dry-etching tuning parameters are known to those skilled in the art.

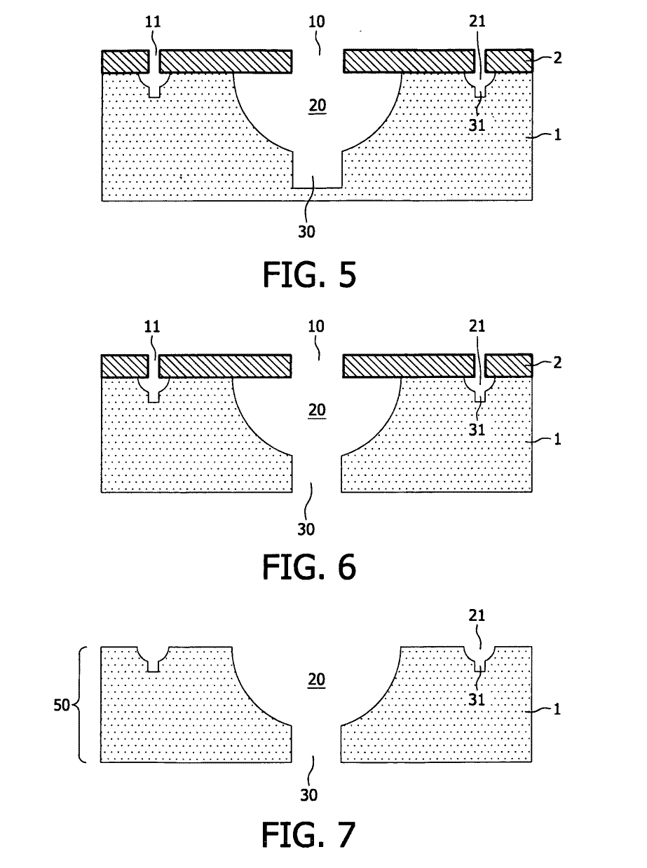

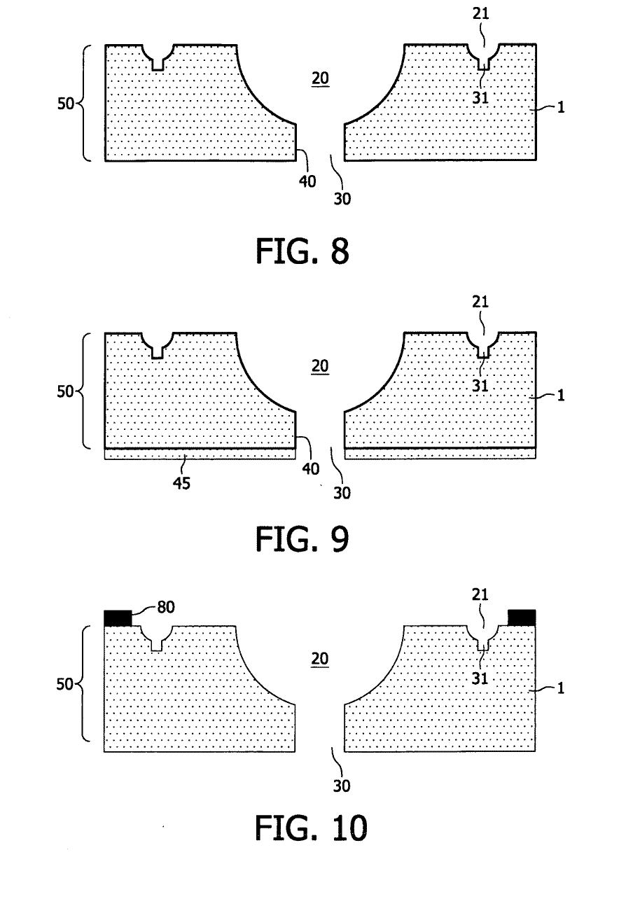

In FIG. 5 the protection layer 3 is removed and in FIG. 6 parts of the silicon substrate 1 are removed from the second side of the substrate 1 by means of grinding or damage etching until the ejection tube is opened. In FIG. 7 the silicon oxide mask layer 2 is removed e.g. by means of buffered oxide etching (BOE) resulting in the structure comprising the ejection chamber 20, the ejection tube 30 and the indentation structure. FIG. 8 shows the additional step of providing a smoothening layer 40. A borophosphosilicate glass (BPSG) is deposited by means of LPCVD on top of the surface of the nozzle 50 as shown in FIG. 7. A subsequent reflow step at around 800° C. further smoothens the surface of the ejection chamber 20 and the ejection tube 30. Additionally, the BPSG smoothening layer 40 is wetted (hydrophilic) by water-based fluids to be ejected. In FIG. 9 a termination layer 45 of octadecyl-trichlorosilane has been evaporated on the second side of the silicon substrate 1 on top of the smoothening layer 40. The termination layer 45 is hydrophobic and is consequently not wetted by means of a water-based fluid to be ejected. This additional measure improves the ejection of the fluid to be ejected by preventing a fluid film on the second side of the substrate 1.

In FIG. 10 a principal sketch of a cross-sectional view of a nozzle 50 as depicted in FIG. 7 is shown with an additional alignment structure 80 on the first side of the silicon substrate 1. The alignment structure 80 is a concentric ring around the ejection chamber 20 and the ejection tube 30 etched in the first side of the silicon substrate 1 before the silicon oxide mask layer 2 is deposited on the first side of the silicon substrate 1.

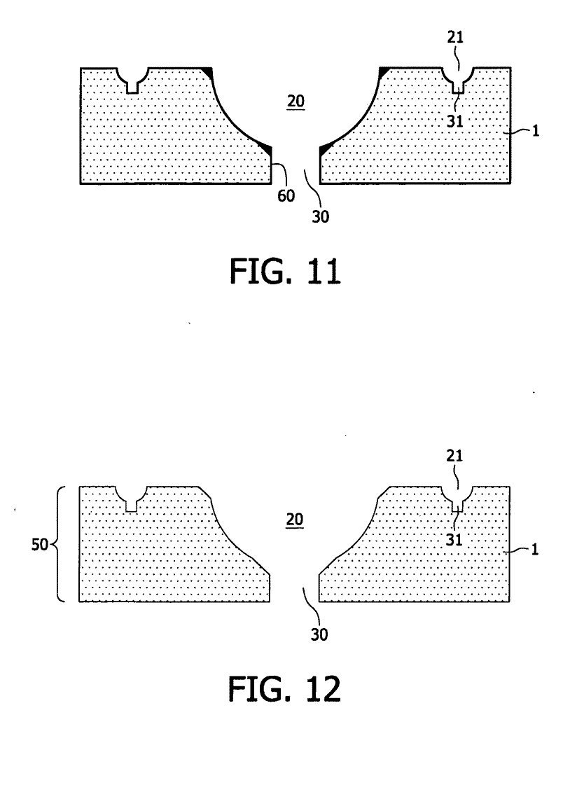

An additional or alternative method to smoothen the surface of the ejection chamber 20 and the ejection tube 30 is shown in FIG. 11 and FIG. 12. In FIG. 11 the surface of the nozzle 50 as depicted in FIG. 7 is thermally oxidized. The silicon oxide layer 60 can cover the whole silicon substrate 1 or the surface of the ejection chamber 20 and the ejection tube 30 if e.g. a patterned Si₃N₄ layer is provided prior to the oxidization step. The oxidization rate depends among other things on the relation between surface and volume. Isolated e.g. spike-like structures and sharp edges are oxidized faster than a flat silicon surface. Consequently, etching of the thermal silicon oxide layer by means of a BOE etch as depicted in FIG. 12 results in rounded edges and a smoothened surface. The advantage of first oxidizing the surface is that this process is well controlled and the silicon oxide layer 60 can be removed in a selective etching process not affecting the remaining silicon.

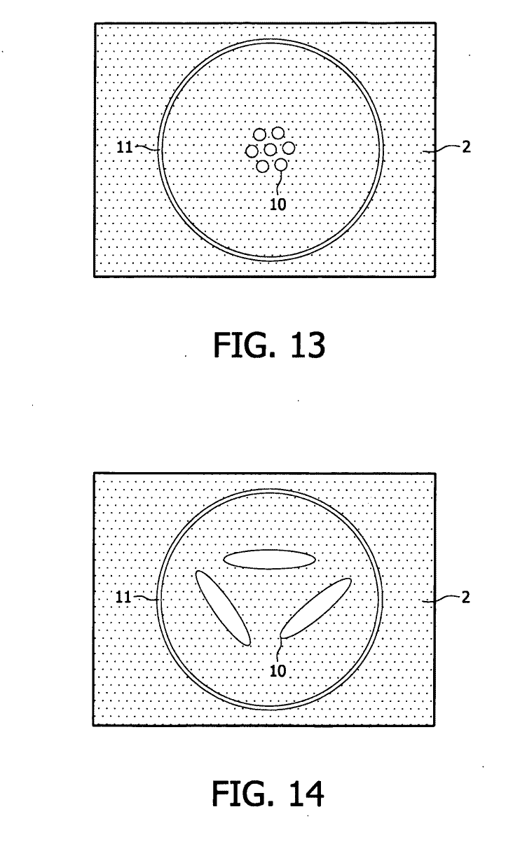

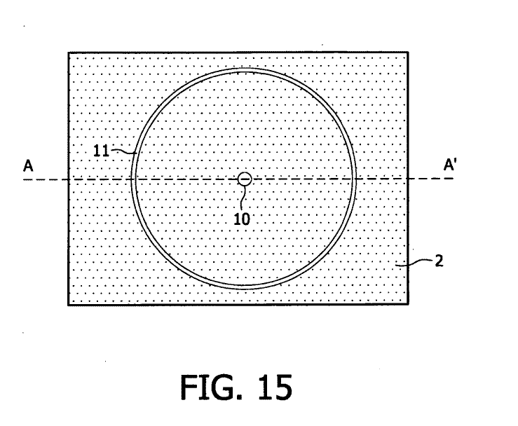

In FIGS. 13 to 15 principal sketches of top views of three different designs of the structured mask layer 2 deposited on the silicon substrate 1 are shown. In FIG. 13 seven round openings 10 surrounded by a concentric circular indentation opening 11 are provided in the mask layer. One of the circular openings 10 is positioned in the center of the indentation opening 11 and is surrounded symmetrically by the remaining six openings 10. Removing the substrate isotropically through the openings 10 results in one common ejection chamber 20 and the subsequent anisotropic removal of the substrate 1 through the openings 10 results in seven adjacent ejection tubes 30 at the bottom of the ejection chamber. In FIG. 14 the round openings 10 of FIG. 13 are replaced by three oval openings 10 arranged symmetrically around the fictive center of the circular indentation opening 11. Again the isotropic removal of the substrate 1 followed by the anisotropic removal of the substrate 1 results in one ejection chamber 20 and three ejection tubes 30. In FIG. 15 one opening 10 is provided in the center of the circular indentation opening 11 and the line AA′ indicates where the cross sections according to FIG. 1-FIG. 12 are made.

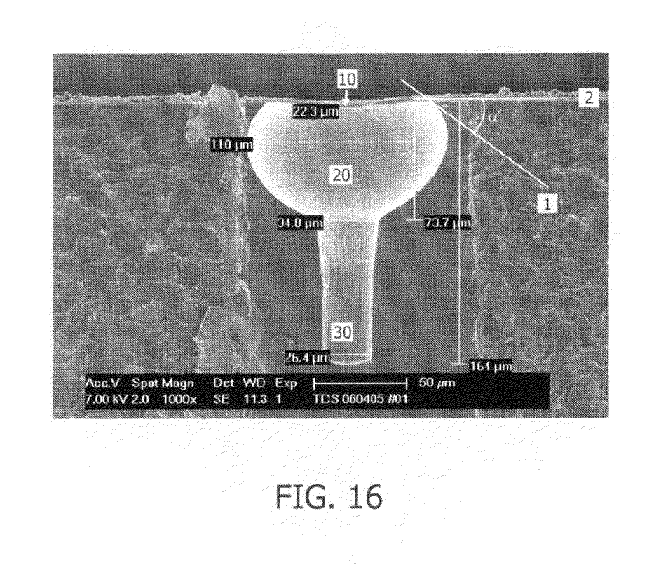

The SEM picture of a nozzle 50 manufactured by means of the method claimed by the current invention, as illustrated in FIG. 16, shows the silicon substrate 1, the mask layer 2 remaining after dry etching with the opening 10, the ejection chamber 20 and the ejection tube 30. The opening 10 is circular and has a diameter of around 22.3 μm. The ejection chamber 20 has a maximum diameter of around 110 μm and the substrate material and the mask layer 2 enclose an angle α of less than 90°, taking the tangent to the boundary of the ejection chamber 20 at the point where the remaining substrate touches the mask layer 2. The ejection tube 30 is cylindrical with a height of around 90 μm and slightly tapering due to process variations with a diameter of around 34 μm at the first opening at the bottom of the ejection chamber 20 and a diameter of around 26.4 μm at the end of the ejection tube 30. In further processing steps the substrate 1 is partly removed from the second side of the substrate 1 by means of etching or grinding in order to provide a second opening of the ejection tube 30. Further, the mask layer 2 is removed and optionally the substrate 1 is partly removed from the first side of the substrate 1 by means of etching or grinding until the maximum diameter of the ejection chamber 20 of around 110 μm is reached.

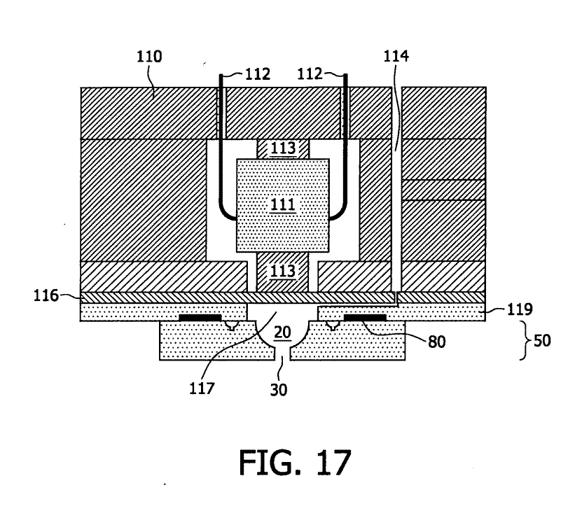

In FIG. 17 a schematic drawing of a nozzle 50 according to the current invention, implemented in a transdermal drug delivery device, is shown. The transdermal drug delivery device comprises the nozzle 50 with a hemispherical ejection chamber 20, a round ejection tube 30, a circular indentation structure and a circular alignment structure 80, a casing 110, a piezoelectric transducer 111 mechanically coupled by a support structure 113 to the casing 110 at a first side and to a membrane 116 at the other side. The piezoelectric transducer 111, for example a small bulk piezoelectric transducer of multilayer ceramic is driven via power lines 112 which connect the piezoelectric transducer 111 to a driving unit (not shown). A microcontroller controls the transdermal drug delivery device, in particular the supply of the piezoelectric transducer 111. The membrane 116 forms a wall of a fluid chamber 117 and the fluid chamber 114 is opened at one side of the ejection chamber and the fluid chamber 117 is connected to a fluid supply line 114. The fluid supply line 114 passes through the membrane 116 at a substantial distance from the fluid chamber 117 and runs at least partly between the membrane 16 and interlayer 119 surrounding the fluid chamber 117. The alignment structure 80 on the first side of the substrate 1 of the nozzle 50 is placed in and adhered to a complementary guide in the interlayer 119. Fluid is supplied to the device via the fluid supply line 114 connected to a fluid reservoir (not shown).

During driving of the piezoelectric transducer 111, the piezoelectric transducer 111 expands and pushes against the flexible membrane 116. This compresses the fluid in the fluid chamber 117, resulting in a pressure buildup, and as a consequence a fluid is focused by the ejection chamber and flows out of the ejection tube 30. As soon as the driving of the piezoelectric transducer 111 stops, both the piezoelectric transducer 111 and the membrane 116 return to their rest states and fluid will enter the fluid chamber 117 and the ejection chamber 20 through the fluid supply line 114 by capillary force.

In order to generate a high-speed fluid ejection, the device is mechanically stiff. If there is too much mechanical deformation of the device during driving of the piezoelectric transducer 111, the pressure in the fluid chamber 117 and the ejection chamber would be too low to generate a high-speed fluid ejection. Further, the relation between the length and the diameter of the fluid supply line 114 has to be high enough in order to apply a sufficiently high pressure to the fluid to be ejected.

Materials that can be used for the construction of the drug delivery device up to the nozzle 50 are stainless steel, aluminum, ceramic, glass, bronze, brass. The device also needs to withstand sterilization procedures. The components can be assembled using two-component epoxy adhesives.

The piezoelectric transducer 111 is driven using a square voltage pulse (or any other suitable shape), which is applied to the piezoelectric transducer 111. In normal operation, the height of the block pulse can vary from 0 to 100 Volts. An increase of the voltage causes an increase of the speed of fluid ejection. The length of the pulse varies between 10 μs and 1000 μs. Increasing the pulse length will influence the volume of the ejected fluid and to a certain extent also the speed. By changing the repetition rate of the block pulse (frequency), the amount of ejected fluid per second can be changed. Common frequencies lie between 1 and 1000 Hz. Again, dosing at low speed requires a square voltage pulse, whereas high-speed ejection in the high-speed regime requires a sudden volume change by a stepwise change in voltage. The fluid chamber 117 and the ejection chamber 30 are self-filling, driven by the surface tension of the fluid, thereby avoiding the need to apply an over-pressure of the fluid reservoir (not depicted). The self-filling of the fluid chamber 117 and the ejection chamber 30 can be further improved by means of surface coatings of the fluid chamber 117 and the ejection chamber 30 being wetted by the fluid to be ejected.

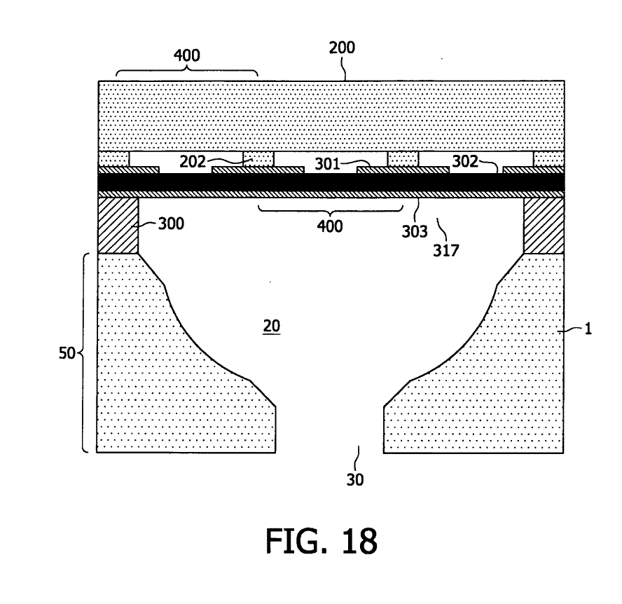

FIG. 18 shows a schematic drawing of a second embodiment of a nozzle 50 according the current invention, implemented in a high-speed ejection device. The high-speed ejection device comprises the nozzle 50, an actuation structure and a support structure. The nozzle 50 comprises a silicon substrate 1, an ejection chamber 20 and an ejection tube 30, both with a smoothened surface according to the description of FIG. 12. The actuation structure comprises a structured silicon base substrate 300, a first electrode layer 303 attached to the base substrate 300, a piezoelectric layer 302, such as e.g. Lead Zirconate Titanate (PZT), deposited on top of the first electrode layer 303 and a structured second electrode layer 301 deposited on top of the piezoelectric layer 302. The support structure comprises a silicon backing substrate 200 and several fixing structures 202, either deposited on the silicon backing substrate 200 or etched in the silicon backing substrate. The actuation structures can be manufactured by well-known semiconductor thin film processing. The high-speed jetting device is assembled by adhering the fixing structures 202 to the electrodes formed by the structured electrode layer 301. The fixing structures 202 provide a mechanical stabilization of the electrodes formed by the structured electrode layer 301 and can in addition be used to provide electrical contacts to the electrodes if the fixing structures comprise an electrically conducting material. The base substrate 300 is partly removed so that independent membranes comprising the first electrode layer 303, the piezoelectric layer 302 and the structured second electrode layer 301 are formed between the fixing structures 202 building an array of piezoelectric membrane transducers 400. The nozzle 50 is adhered to the residues of the base substrate 300 in a way that the array of membrane transducers faces the ejection chamber 20, and the residues of the base substrate 300 and the membranes of the membrane transducer 400 bound the fluid chamber 317. The ejection chamber 20 and the fluid chamber 317 form one common cavity that can be filled with a fluid to be ejected. The high-speed ejection device with the nozzle 50 according to the current invention can be manufactured in a wafer-based semiconductor process. A multitude of high-speed ejection devices can be processed in parallel by means of a three-wafer process comprising a wafer with the nozzles 50, a wafer with actuation structures and a wafer with the support structures. Further, an array of several high-speed ejection devices can be easily manufactured. The latter might be advantageous if the high-speed ejection device is used for transdermal drug delivery in order to prevent irritation of the skin.

The present invention will be described with respect to particular embodiments and with reference to certain drawings, but this is not to be construed in a limiting sense, as the invention is limited only by the appended claims. Any reference signs in the claims shall not be construed as limiting the scope thereof. The drawings described are only schematic and are non-limiting. In the drawings, the size of some of the elements may be exaggerated and not drawn to scale for illustrative purposes. Where the term “comprising” is used in the present description and claims, it does not exclude other elements or steps. Where an indefinite or definite article is used when referring to a singular noun, e.g. “a” or “an”, “the”, this includes a plural of that noun unless specifically stated otherwise.

Furthermore, the terms first, second, third and the like in the description and in the claims are used for distinguishing between similar elements and not necessarily for describing a sequential or chronological order. It is to be understood that the terms so used are interchangeable under appropriate circumstances, and that the embodiments of the invention described herein are capable of operation in other sequences than described or illustrated herein.

Moreover, the terms top, bottom, first, second and the like in the description and the claims are used for descriptive purposes and not necessarily for describing relative positions. It is to be understood that the terms so used are interchangeable under appropriate circumstances and that the embodiments of the invention described herein are capable of operation in other orientations than described or illustrated herein.

1. Method of manufacturing a nozzle (50) for jetting of fluids comprising the steps of:
providing a substrate (1) having a first side and a second side;
providing a mask layer (2) on the first side of the substrate (1) and a protection layer (3) on the second side of the substrate (1);
providing at least one opening (10) in the mask layer (2);
forming an ejection cavity (20) by removing parts of the substrate (1) isotropically through the opening (10) in the mask layer (2);
forming at least one ejection tube (30) by removing parts of the substrate (1) anisotropically through the opening (10) in the mask layer (2),
opening the ejection tube at the second side of the substrate (1), and
removing the mask layer (2).

2. Method according to claim 1, comprising the additional step of removing a part of the substrate (1) from the first side of the substrate (1) after removing the mask layer (2).

3. Method according to claim 1, comprising the additional step of smoothening the surface of the ejection cavity (20) and smoothening the surface of the ejection tube (30) by removing the surface of the ejection cavity (20) and the ejection tube (30) superficially.

4. Method according to claim 1, comprising the additional step of providing a smoothening layer (40) at least on the surface of the ejection cavity (20) and the ejection tube (30).

5. Method according to claim 4, wherein the smoothening layer (40) consists of a material having an angle of contact with a fluid to be ejected of less than 90°.

6. Method according to claim 1, comprising the additional step of providing a termination layer (45) on the second surface of the substrate (1), which termination layer (45) consists of a material having an angle of contact with the fluid to be ejected of more than 90°.

7. Method according to claim 1, wherein at least one further indentation structure (21, 31) is provided in the first side of the substrate (1).

8. Method according to claim 1, comprising the additional step of providing an alignment structure (80) on the first side of the substrate (1).

9. A nozzle (50) for jetting of fluids, comprising:
a substrate (1) with a first side and a second side;
an ejection chamber (20) in the substrate (1), which is open on the first side of the substrate (1), wherein the cross-section of the ejection chamber (20) parallel to the first side of the substrate (1) is not constant as a function of depth; and
at least one ejection tube (30) in the substrate (1) open on the second side of the substrate (1), wherein the cross-section of the ejection tube (30) parallel to the first side of the substrate (1) is constant as a function of depth, and
wherein the ejection chamber (20) and the at least one ejection tube (30) are connected to form a passage through the substrate (1).

10. A nozzle (50) according to claim 9, wherein the ejection tube (30) is cylindrical, the ejection chamber (20) is a hemispherical cavity, and the ejection chamber (20) and the ejection tube (30) are aligned along the cylinder axis of the ejection tube (30).

11. A jetting device comprising a nozzle (50) according to claim 9.

12. A jetting device according to claim 11, comprising a power supply, a pressure applicator adapted for applying a pressure to a fluid to be ejected through the nozzle, and the second side of the substrate (1) of the nozzle (50) is part of the outer surface of the jetting device.

13. A jetting device according to claim 11, wherein said device is configured for transdermal drug delivery.
